(19) United States
(12) Plant Patent Application Publication
(10) Pub. No.: US 2015/0181793 P1
(43) Pub. Date: Jun. 25, 2015

(54) New Guinea Impatiens plant named 'SAKIMP031'

(50) ;

(71) Applicant: Sakata Seed Corporation, Yokohama (JP)

(72) Inventors: Chihiro Sato, Nagano (JP); Shinji Minemura, Nagano (JP)

(73) Assignee: Sakata Seed Corporation, Yokohama (JP)

(21) Appl. No.: 13/998,937

(22) Filed: Dec. 23, 2013

(51) Int. Cl. A01H 5/00

(52) U.S. Cl. PLT/318.4

(57) ABSTRACT

A New Guinea Impatiens plant particularly distinguished by having a pink flower color, strong root system and a spreading plant growth habit, is disclosed.

### GENUS AND SPECIES

Impatiens hybrida hort

### VARIETY DENOMINATION

‘SAKIMP031’

### BACKGROUND OF THE NEW PLANT

The present invention comprises of a new and distinct variety of New Guinea Impatiens, botanically known as Impatiens hybrida hort, and referred to by the variety name ‘SAKIMP031’. ‘SAKIMP031’ originated from a hybridization in September 2009 in Misato, Japan between the proprietary female Impatiens breeding line ‘NE-4391’ (unpatented) having a blush flower color and the proprietary male Impatiens breeding line ‘NF-4858’ (unpatented) having a red flower color.

In September 2009, the female parent line ‘NE-4391’ and male parent line ‘NF-4858’ were crossed and a population of F₁ plants was created. The F₁ plants were evaluated in Misato, Japan in an open field trial. The criteria for plant selection included flower color, a strong root system and a spreading plant growth habit. At the completion of the trial, one single-plant selection was made based on the above criteria and vegetatively propagated. From May to August 2011, the selection was evaluated in an open field in Misato, Japan. Terminal tip vegetative cuttings of the variety were then shipped to Salinas, Calif., where the plants were regenerated and reevaluated for stability of traits. The selection subsequently was named ‘SAKIMP031’ and was found to have its unique characteristics reproduce true to type in successive generations of asexual propagation via terminal tip vegetative cuttings in Salinas, Calif.

### SUMMARY

The following are the most outstanding and distinguishing characteristics of this new variety when grown under normal horticultural practices in Salinas, Calif.

1. Pink flowers;

2. Strong root system; and

3. A spreading plant growth habit.

### DESCRIPTION OF THE PHOTOGRAPHS

This Impatiens plant is illustrated by the accompanying photographs which show the plant's overall plant habit including form, foliage, and flowers. The photographs are of plants grown about 4-months-old in Salinas, Calif. under greenhouse conditions in the spring of 2013. The colors shown are as true as can be reasonably obtained by conventional photographic procedures.

FIG. 1 shows a close-up of the mature inflorescence of the plant.

FIG. 2 shows the overall plant habit of the plant grown in a pot.

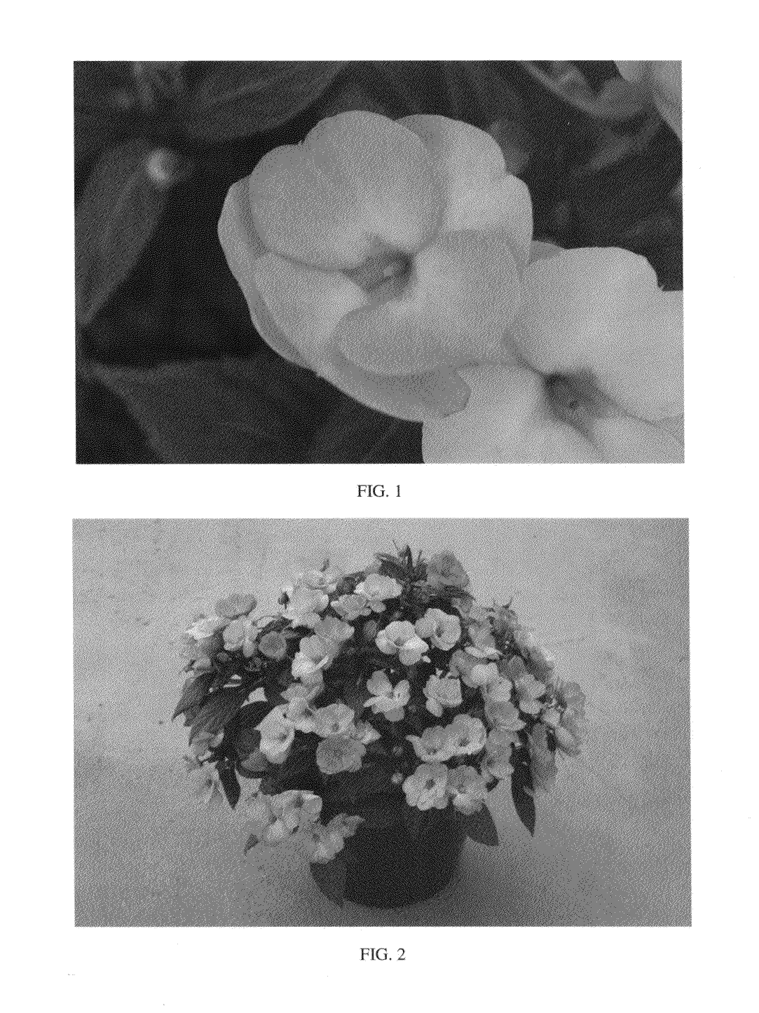

### DESCRIPTION OF THE NEW VARIETY

The following detailed descriptions set forth the distinctive characteristics of ‘SAKIMP031’. The data which define these characteristics were collected from asexual reproductions carried out in Salinas, Calif. Data was collected on plants grown about 1 month from transplant date (about 4-months-old) and propagated by terminal tip vegetative cuttings under greenhouse conditions in Salinas, Calif. in the summer of 2013. Color references are to The R.H.S. Colour Chart of The Royal Horticultural Society of London (R.H.S.), 4^{th} edition (2001).
Classification:
   Family.—Balsaminaceae.
   Botanical.—Impatiens interspecific cross (Impatiens hybrida hort).
   Common.—Impatiens.
   Designation.—‘SAKIMP031’.
Parentage:
   Female parent.—Proprietary Impatiens line ‘NE-4391’ (unpatented).
   Male parent.—Proprietary Impatiens line ‘NF-4858’ (unpatented).
Growth:
   Time to produce a rooted cutting.—About 4 weeks.
   Environmental conditions for plant growth.—The terminal 1.0 to 1.5 inches of an actively growing stem was excised. The vegetative cuttings were propagated in five to six weeks. The base of the cuttings were dipped for 1 to 2 seconds in a 1:9 solution of DIP 'N GROW (1 solution: 9 water) a root inducing solution, immediately prior to sticking into the cell trays. Cuttings were stuck into plastic cell trays having 98 cells, and containing a moistened peat moss-based growing medium. The cuttings were misted with water from overhead for 10 seconds every 30 minutes until sufficient roots were formed. Rooted cuttings were transplanted and grown in 20-cm-diameter plastic pots in a glass greenhouse located in Salinas, Calif. Pots contained a peat moss-based growing medium. Soluble fertilizer containing 20% nitrogen, 10% phosphorus and 20% potassium was applied once a day or every other day by overhead irrigation. Pots were top-dressed with a dry, slow release fertilizer containing 20% nitrogen, 10% phosphorus and 18% potassium. The typical average air temperature was 24° C.
   Time to bloom from propagation.—6 to 8 weeks.
Plant description:
   Habit.—Spreading.
   Life cycle.—Annual.
   Height (from soil line to top of foliage).—24.0 cm.
   Height (from soil line to first node).—1.0 cm.
   Spread.—45.0 cm.
   Flowering requirements.—Will flower so long as temperature is above 5° C.
   Temperature tolerances.—Plants observed to continue flowering in a temperature range of 5° C. to 36° C. Plants can withstand high heat and humidity.
Branches:
   Number of branches.—6 main branches.
   General description.—Smooth, circular cross-section.
   Length of branches.—9.0 cm.
   Diameter of branches.—1.0 cm.
   Internodes.—4.0 cm.
   Color of branches.—RHS 59A (Red-Purple) with RHS 144B (Yellow-Green).
   Anthocyanin color.—RHS 59A.
   Pubescence.—Absent.
Stems:
   General description.—Strong; circular cross-section, smooth.
   Stem length.—11.0 cm.
   Diameter.—0.5 cm.
   Internode length.—7.0 cm.
   Color.—RHS 59B (Red-Purple) with slight RHS 144B (Yellow-Green).
   Pubescence.—Absent.
   Anthocyanin color.—RHS 59B (Red-Purple).
Leaves:
   Arrangement.—Opposite.
   Shape.—Lanceolate.
   Apex.—Acute.
   Base.—Attenuate.
   Margin.—Entire.
   Surface appearance (both surfaces).—Dull.
   Surface pubescence (both surfaces).—Absent; surface is glabrous.
   Length.—9.0 cm.
   Width.—3.0 cm.
   Color.—Upper surface: RHS 147A (Yellow-Green) Lower surface: RHS 147B (Yellow-Green).
   Venation.—Pinnate.
   Venation color (both surfaces).—RHS 59D (Red-Purple).
   Variegation.—Absent.
   Fragrance.—Absent.
   Petiole.—Length: 1.0 cm Diameter: 0.3 cm.
Flowers:
   Total number of flowers per node.—2 to 3 in bloom.
   Inflorescence type.—Single flower with spur and 5 petals, floriferous.
   Fragrance.—Absent.
Flower bud:
   Surface texture.—Smooth, glabrous.
   Length.—1.5 cm.
   Diameter.—1.3 cm.
   Shape.—Deltoid.
   Color.—RHS 59B (Red-Purple) with RHS 58B (Red-Purple) and slight RHS 144A (Yellow-Green) at the edge.
Peduncle:
   Length.—4.0 cm.
   Width.—0.2 cm.
   Color.—RHS 59C (Red-Purple).
   Surface texture.—Glabrous, smooth.
Corolla:
   Shape.—Rotate, stellate.
   Diameter.—5.0 cm.
   Depth.—2.0 cm.
   Petals.—Shape: Bi-lobed, curved, recessed at mid-vein Apex: Obcordate Margin: Entire Pubescence (both surfaces): Glabrous Length: 2.0 cm Width: 3.0 cm Color: Upper: RHS 58C (Red-Purple) with some RHS 58D (Red-Purple) Lower: RHS 58C (Red-Purple) with some RHS 58C (Red-Purple) and RHS 59C (Red-Purple) at the mid-veins Eye zone: RHS 64C (Red-Purple).
Spur:
   Shape.—Horn-shaped.
   Length.—4.0 cm.
   Diameter.—0.2 cm.
   Color.—RHS 59C (Red-Purple).
   Texture.—Smooth, glabrous.
Calyx:
   Arrangement.—Composed of 2 sepals.
   Sepals.—Shape: Lanceolate, curled Apex: Acute Base: Obtuse Margin: Entire Surface texture: Smooth, glabrous Color: RHS 62B (Red-Purple) Length: 2.0 cm Diameter: 1.0 cm.
Reproductive organs:
   Stamen number.—5.
   Stamen form.—Fused on top of ovary.
   Filament color.—RHS 58C (Red-Purple).
   Anther color.—RHS 155A (White).
   Pollen color.—RHS 155D (White).
   Pollen amount.—Abundant.
   Ovary arrangement.—Fused in capsule.
   Ovary surface color.—RHS 144A (Yellow-Green).
   Ovary shape.—Ovate.
   Pistil number.—1.
   Stigma color and shape.—RHS 155A (White) and hair-like.
   Style color.—RHS 155A (White).
   Seed production.—Absent.
Disease and insect resistance: Tolerant to downy mildew

### COMPARISON WITH PARENTAL LINES AND KNOWN VARIETY

‘SAKIMP031’ is a new and unique variety of Impatiens owing to its pink flower color, strong root system and a compact plant growth habit. ‘SAKIMP031’ is distinguished from its parents mainly by flower color as shown in Table 1 below:

TABLE 1
Comparison with Parental Lines

| | | Female Parent | Male Parent |
|---|---|---|---|
| Characteristic | ‘SAKIMP031’ | ‘NE-4391’ | ‘NF-4858’ |
| Flower color | Pink | Blush | Red |

‘SAKIMP031’ is a new and unique variety of Impatiens owing to its pink flower color, strong root system and a compact plant growth habit. When ‘SAKIMP031’ compared to the commercial Impatiens variety ‘SAKIMP022’ (U.S. Plant Pat. No. 23,500), the following differences as described in Table 2 below.

TABLE 2
Comparison with Similar Variety

| Characteristic | ‘SAKIMP031’ | ‘SAKIMP022’ |
|---|---|---|
| Petal color, upper surface | RHS 58C with some RHS 58D | RHS N57A |
| Petal color, lower surface | RHS 58C with some RHS 58C | RHS 58B |
| | and RHS 59C at the mid-vein | |
| Petal color, eye zone | RHS 64C | RHS 60A |
| Stem color | RHS 59B with RHS 144B | RHS 187B |

We claim:

1. A new and distinct variety of New Guinea Impatiens plant as shown and described herein.
